SEQUENCE LISTING (11 sequences)

SEQ ID NO: 1 - protein, 50 aa, Araneus diadematus
SGCDVLVQALLEVVSALVSILGSSSIGQINYGASAQYTQMVGQSVAQALA

SEQ ID NO: 2 - protein, 30 aa, Araneus diadematus
SGCDVLVQALLEVVSALVSILGSSSIGQIN

SEQ ID NO: 3 - protein, 21 aa, Araneus diadematus
SGCDVLVQALLEVVSALVSIL

SEQ ID NO: 4 - protein, 24 aa, Artificial Sequence
  His tag and start codon
MHHHHHHHHHHSSGSSLEVLFQGP

SEQ ID NO: 5 - protein, 660 aa, Artificial Sequence
  recombinant spider silk protein ADF3Kai
MHHHHHHHHHHSSGSSLEVLFQGPARAGSGQQGPGQQGPGQQGPGQQGPYGPGASAAAAAAGGYGPGSGQQGPSQQGPGQQGPGGQGPYGPGASAAAAAAGGYGPGSGQQGPGGQGPYGPGSSAAAAAAGGNGPGSGQQGAGQQGPGQQGPGASAAAAAAGGYGPGSGQQGPGQQGPGGQGPYGPGASAAAAAAGGYGPGSGQGPGQQGPGGQGPYGPGASAAAAAAGGYGPGSGQQGPGQQGPGQQGPGGQGPYGPGASAAAAAAGGYGPGYGQQGPGQQGPGGQGPYGPGASAASAASGGYGPGSGQQGPGQQGPGGQGPYGPGASAAAAAAGGYGPGSGQQGPGQQGPGQQGPGQQGPGGQGPYGPGASAAAAAAGGYGPGSGQQGPGQQGPGQQGPGQQGPGQQGPGQQGPGQQGPGQQGPGQQGPGGQGAYGPGASAAAGAAGGYGPGSGQQGPGQQGPGQQGPGQQGPGQQGPGQQGPGQQGPGQQGPYGPGASAAAAAAGGYGPGSGQQGPGQQGPGQQGPGGQGPYGPGAASAAVSVGGYGPQSSSVPVASAVASRLSSPAASSRVSSAVSSLVSSGPTKHAALSNTISSVVSQVSASNPGLSGCDVLVQALLEVVSALVSILGSSSIGQINYGASAQYTQMVGQSVAQALA

SEQ ID NO: 6 - DNA, 1983 bp, Artificial Sequence
  recombinant DNA of spider silk protein gene ADF3Kai
atgcatcaccatcatcatcatcaccaccaccattcctcgggctcatccttggaagtgttatttcaaggaccagcacgagccggttcgggacaacaagggcctggccagcagggcccaggtcaacaagggccaggacagcagggtccttatgggcccggcgcaagcgcagcagctgcggccgctggtggctatggtcctggctccggtcaacagggcccttcgcaacaaggtcccgggcagcaaggtcctggtggccagggtccctacgggccgggggcgagtgcggcagcagccgctgcaggcggttatggtccaggaagcggacagcaaggtccgggaggtcaaggtccgtatggcccaggctctagcgcggctgccgctgccgcgggtggcaacggaccagggagcggacaacagggcgcgggacaacagggtccaggacagcaaggcccaggggcgtcggcggctgcagcggcggccggaggctatggacccggctcaggacaacagggaccgggtcaacaaggacccggtggccaaggcccctatggcccgggcgccagcgcggccgcagccgccgcgggcgggtacggccccggtagcggccagggaccaggtcagcaggggccaggaggtcagggcccatacggtccgggcgcatccgcggcggcggcagcggcaggtggctacggtcccggaagcggccaacaggggccagggcaacaaggaccaggacaacaaggtcctgggggccaaggaccgtatggaccaggagcatcagctgcagccgcggcagctggcggttacggtccaggctacggccagcagggtccgggtcagcagggaccgggaggccaggggccttatggccctggcgcttccgcagccagtgccgcttctggaggatacgggccgggaagcggtcagcaaggccctggccaacaaggacctggaggccaagggccctacggcccaggagcctcggcagccgcagctgccgcaggtgggtatgggccaggtagcgggcaacaagggccgggtcagcaaggaccggggcaacagggacctgggcagcaaggacccgggggtcaaggcccgtacggacctggtgcgtctgcagctgctgctgcggctggtggatatggtccgggatcggggcagcagggtcccggtcagcagggccctggtcagcaagggccaggccaacagggacccggacaacaaggcccgggtcaacagggtcctggacagcaggggccgggccaacaaggccctgggcaacagggtccggggggacagggggcctatgggcctggcgcatctgccgccgctggcgcagccggtgggtacgggcctgggtcaggtcaacaggggcctggtcaacaaggccccgggcaacagggccccggccagcaaggtccagggcagcagggcccgggacagcaagggcctggacaacaggggcccggacagcagggaccttacgggcccggtgcgagcgcagcggccgccgccgcagggggatatggccccggatcgggccagcagggaccaggccagcaaggacctggccaacagggcccggggggtcaggggccgtatggtcccggcgctgcaagtgctgcagtgtccgttggaggttacggccctcagtcttcgtctgttccggtggcgtccgcagttgcgagtagactgtcttcacctgctgcttcatcgcgagtatcgagcgctgtttcgtctcttgtctcgtcgggtcccacgaaacatgccgccctttcaaatacgatttcatctgtagtgtcccaagttagtgcaagtaacccggggttatccggatgcgacgttctcgttcaggcactcctagaagtagtatccgcgttggtgagcatcttaggcagctcctcgataggtcaaataaactatggtgcttcagcccagtatacacagatggtgggacagagcgtcgcgcaggcattggcttaa

SEQ ID NO: 7 - protein, 542 aa, Artificial Sequence
  recombinant DNA of spider silk protein gene ADF3Kai
MHHHHHHHHHHSSGSSLEVLFQGPARAGSGQQGPGQQGPGQQGPGQQGPYGPGASAAAAAAGGYGPGSGQQGPSQQGPGQQGPGGQGPYGPGASAAAAAAGGYGPGSGQQGPGGQGPYGPGSSAAAAAAGGNGPGSGQQGAGQQGPGQQGPGASAAAAAAGGYGPGSGQQGPGQQGPGGQGPYGPGASAAAAAAGGYGPGSGQGPGQQGPGGQGPYGPGASAAAAAAGGYGPGSGQQGPGQQGPGQQGPGGQGPYGPGASAAAAAAGGYGPGYGQQGPGQQGPGGQGPYGPGASAASAASGGYGPGSGQQGPGQQGPGGQGPYGPGASAAAAAAGGYGPGSGQQGPGQQGPGQQGPGQQGPGGQGPYGPGASAAAAAAGGYGPGSGQQGPGQQGPGQQGPGQQGPGQQGPGQQGPGQQGPGQQGPGQQGPGGQGAYGPGASAAAGAAGGYGPGSGQQGPGQQGPGQQGPGQQGPGQQGPGQQGPGQQGPGQQGPYGPGASAAAAAAGGYGPGSGQQGPGQQGPGQQGPGGQGPYGPGAASAA

SEQ ID NO: 8 - protein, 434 aa, Artificial
  Recombinang spider silk protein ADF4Kai
MHHHHHHHHHHSSGSSLEVLFQGPAGSSAAAAAAASGSGGYGPENQGPSGPVAYGPGGPVSSAAAAAAAGSGPGGYGPENQGPSGPGGYGPGGSGSSAAAAAAAASGPGGYGPGSQGPSGPGGSGGYGPGSQGASGPGGPGASAAAAAAAAAASGPGGYGPGSQGPSGPGAYGPGGPGSSAAAAAAAASGPGGYGPGSQGPSGPGVYGPGGPGSSAAAAAAAGSGPGGYGPENQGPSGPGGYGPGGSGSSAAAAAAAASGPGGYGPGSQGPSGPGGSGGYGPGSQGGSGPGASAAAAAAAASGPGGYGPGSQGPSGPGYQGPSGPGAYGPSPSASASVAASVYLRLQPRLEVSSAVSSLVSSGPTNGAAVSGALNSLVSQISASNPGLSGCDALVQALLELVSALVAILSSASIGQVNVSSVSQSTQMISQALS

SEQ ID NO: 9 - protein, 355 aa, Artificial
  Recombinant spider silk protein MaSp2_N
MHHHHHHSSGSSLEVLFQGPARAGPGGYRPGQQGPSGPGSAAAAAAAAAGPGGYGPGQQGPGGYGPGQQGPSGAGSAAAAAAAGPGQQGLGGYGPGQQGPGGYGPGQQGPGGYGPGSASAAAAAAGPGQQGPGGYGPGQQGPSGPGSASAAAAAAGPGGYGPGQQGPGGYAPGQQGPSGPGSAAAAAAAARAGPGGYGPAQQGPSGPGIAASAASAGPGGYGPAQQGPAGYGPGSAVAASAGAGSAGYGPGSQASAAASRLASPDSGARVASAVSNLVSSGPTSSAALSSVISNAVSQIGASNPGLSGCDVLIQALLEIVSACVTILSSSSIGQVNYGAASQFAQVVGQSVLSAF

SEQ ID NO: 10 - protein, 559 aa, Artificial
  Recombinant spider silk protein Flag_92_short2
MHHHHHHHHHHSSGSSLEVLFQGPGAGGSGPGGAGPGGVGPGGSGPGGVGPGGSGPGGVGPGGSGPGGVGPGGAGGPYGPGGSGPGGAGGAGGPGGAYGPGGSYGPGGSGGPGGAGGPYGPGGEGPGGAGGPYGPGGAGGPYGPGGAGGPYGPGGEGGPYGPGGSYGPGGAGGPYGPGGPYGPGGEGPGGAGGPYGPGGVGPGGGGPGGYGPGGAGPGGYGPGGSGPGGYGPGGSGPGGYGPGGSGPGGYGPGGSGPGGYGPGGSGPGGSGPGGYGPGGSGPGGSGPGGYGPGGSGPGGYGPGGSGPGGSGPGGYGPGGSGPGGSGPGGYGPGGSGPGGFGPGGFGPGGSGPGGYGPGGSGPGGAGPGGVGPGGFGPGGAGPGGAGPGGAGPGGAGPGGAGPGGAGPGGAGPGGAGPGGAGGAGGAGGAGGSGGAGGSGGTTIIEDLDITIDGADGPITISEELTISAYYPSSRVPDMVNGIMSAMQGSGFNYQMFGNMLSQYSSGSGTCNPNNVNVLMDALLAALHCLSNHGSSSFAPSPTPAAMSAYSNSVGRMFAY

SEQ ID NO: 11 - DNA, 1629 bp, Artificial Sequence
  recombinant spider silk protein gene ADF3Kai-noNR
atgcatcaccatcatcatcatcaccaccaccattcctcgggctcatccttggaagtgttatttcaaggaccagcacgagccggttcgggacaacaagggcctggccagcagggcccaggtcaacaagggccaggacagcagggtccttatgggcccggcgcaagcgcagcagctgcggccgctggtggctatggtcctggctccggtcaacagggcccttcgcaacaaggtcccgggcagcaaggtcctggtggccagggtccctacgggccgggggcgagtgcggcagcagccgctgcaggcggttatggtccaggaagcggacagcaaggtccgggaggtcaaggtccgtatggcccaggctctagcgcggctgccgctgccgcgggtggcaacggaccagggagcggacaacagggcgcgggacaacagggtccaggacagcaaggcccaggggcgtcggcggctgcagcggcggccggaggctatggacccggctcaggacaacagggaccgggtcaacaaggacccggtggccaaggcccctatggcccgggcgccagcgcggccgcagccgccgcgggcgggtacggccccggtagcggccagggaccaggtcagcaggggccaggaggtcagggcccatacggtccgggcgcatccgcggcggcggcagcggcaggtggctacggtcccggaagcggccaacaggggccagggcaacaaggaccaggacaacaaggtcctgggggccaaggaccgtatggaccaggagcatcagctgcagccgcggcagctggcggttacggtccaggctacggccagcagggtccgggtcagcagggaccgggaggccaggggccttatggccctggcgcttccgcagccagtgccgcttctggaggatacgggccgggaagcggtcagcaaggccctggccaacaaggacctggaggccaagggccctacggcccaggagcctcggcagccgcagctgccgcaggtgggtatgggccaggtagcgggcaacaagggccgggtcagcaaggaccggggcaacagggacctgggcagcaaggacccgggggtcaaggcccgtacggacctggtgcgtctgcagctgctgctgcggctggtggatatggtccgggatcggggcagcagggtcccggtcagcagggccctggtcagcaagggccaggccaacagggacccggacaacaaggcccgggtcaacagggtcctggacagcaggggccgggccaacaaggccctgggcaacagggtccggggggacagggggcctatgggcctggcgcatctgccgccgctggcgcagccggtgggtacgggcctgggtcaggtcaacaggggcctggtcaacaaggccccgggcaacagggccccggccagcaaggtccagggcagcagggcccgggacagcaagggcctggacaacaggggcccggacagcagggaccttacgggcccggtgcgagcgcagcggccgccgccgcagggggatatggccccggatcgggccagcagggaccaggccagcaaggacctggccaacagggcccggggggtcaggggccgtatggtcccggcgctgcaagtgctgcataa

(19) United States
(12) Patent Application Publication
(10) Pub. No.: US 2018/0251501 A1
(43) Pub. Date: Sep. 6, 2018

(54) Molded Article and Method for Producing Molded Article

(71) Applicants: Spiber Inc., Yamagata (JP); Kojima Industries Corporation, Aichi (JP); Tekunohama Co., Ltd., Aichi (JP)

(72) Inventors: Junichi Sugahara, Yamagata (JP); Kaori Sekiyama, Yamagata (JP); Ayumi Abe, Yamagata (JP); Junichi Shimokata, Yamagata (JP); Junichi Noba, Aichi (JP); Shinji Hirai, Hokkaido (JP)

(73) Assignees: Spiber Inc., Yamagata (JP); Kojima Industries Corporation, Aichi (JP); Tekunohama Co., Ltd., Aichi (JP)

(21) Appl. No.: 15/760,492

(22) PCT Filed: Sep. 8, 2016
(86) PCT No.: PCT/JP2016/076500

§ 371 Date: Mar. 15, 2018

(30) Foreign Application Priority Data
Sep. 18, 2015 (JP) 2015-185766

(51) Int. Cl. C07K 14/435; B29C 43/52

(52) U.S. Cl. CPC C07K 14/43518; B29K 2089/00; B29C 43/52

(57) ABSTRACT

Provided is a molded article of a composition comprising a polypeptide which has more than 4.3 GPa of a bending elastic modulus and transparency.

### TECHNICAL FIELD

The present invention relates to a molded article and a method for producing a molded article.

### BACKGROUND ART

Inorganic glass is excellent in terms of transparency or thermal resistance and is thus used as window members in a number of uses including car uses. On the other hand, inorganic glass is poor in terms of formability to three-dimensional curves and has a great specific weight, and thus synthetic resin windows made of an acrylic resin or a polycarbonate resin which does not easily cause the above-described problems have been proposed (for example, refer to Patent Literature 1). In addition, due to a rising demand for environmental protection, bioplastics which are petroleum-free materials have been thus far drawing attention, as a transparent and high-strength material, “TERRAMAC” (registered trademark) is known, and transparent materials having 99.5% of a polylactic acid percentage and 4.3 GPa of a bending elastic modulus have been realized.

### CITATION LIST

### Patent Literature

[Patent Literature 1] JP H6-156071 A

### SUMMARY OF INVENTION

### Technical Problem

Regarding car window glass, the weight has an influence on gas mileage and, furthermore, may cause a problem of environmental burden, and thus weight reduction is extremely important. Therefore, an object of the present invention is to provide a highly transparent formed article which can weigh less than conventional synthetic resins and a production method therefor.

### Solution to Problem

The present invention provides a molded article of a composition comprising a polypeptide wherein the molded article has more than 4.3 GPa of a bending elastic modulus and transparency.

The molded article has transparency and thus can be applied to window uses with no problems. In addition, the molded article exhibits a bending elastic modulus as high as more than 4.3 GPa, and thus, in the case of being formed to a panel shape, the molded article is capable of producing a strength that is equal to or higher than those of conventional synthetic resins with a thinner thickness, which significantly contributes to weight reduction. The specific weight of the molded article is also equal to those of synthetic resins such as acrylic resins or polycarbonate resins and is overwhelmingly lighter than that of inorganic glass. Therefore, in a number of uses including car uses, it is possible to reduce environmental burden by weight reduction. Meanwhile, the bending elastic modulus is preferably 4.7 GPa or more, more preferably 5.0 GPa or more, and still more preferably 5.2 GPa or more. The upper limit of the bending elastic modulus is not limited, but can be set to, for example, 15.0 GPa or less. The bending elastic modulus can be set to 10.0 GPa or less and, furthermore, 7.0 GPa or less.

As the polypeptide, the composition preferably contains at least one kind selected from the group consisting of natural spider silk protein and polypeptides derived from natural spider silk protein. The natural spider silk protein or the polypeptide derived from natural spider silk protein is significantly excellent in terms of both the bending elastic modulus and the bending strength and, when added as the polypeptide, enables additional weight reduction and additional function improvement.

As the polypeptide, the composition may further contain silk fibroin, soybean protein, casein, keratin, collagen, and milk whey protein. When these are jointly used with the natural spider silk protein or the polypeptide derived from natural spider silk protein, it becomes easy to design the performance of the molded article depending on intended uses.

The molded article may be provided as a hot press-molded article. The molded article refers to an article or the like formed using a casting mold (mold), but articles having a superior bending elastic modulus can be produced by hot pressing procedure.

The molded article can be produced using a production method comprising a step of hot pressing the above-described composition comprising the polypeptide.

### Advantageous Effects of Invention

According to the present invention, a highly transparent formed article which can weigh less than synthetic resins of the related art and a production method therefor are provided.

### BRIEF DESCRIPTION OF DRAWINGS

FIG. 1 is a schematic cross-sectional view of a press molding machine.

FIG. 2 illustrates schematic cross-sectional views of the press molding machine (a) before the introduction of a composition, (b) immediately after the introduction of the composition, and (c) with the composition in a state of being hot pressed.

FIG. 3 is a photograph of a molded article of Example 1.

### DESCRIPTION OF EMBODIMENTS

Hereinafter, a preferred embodiment of the present invention will be described. However, the present invention is not limited to the following embodiment by any means.

A molded article according to the embodiment is made of a composition comprising a polypeptide and the polypeptide preferably includes natural spider silk protein and/or a polypeptide derived from natural spider silk protein (hereinafter, in some cases, these will be collectively referred to as “spider silk polypeptide”). The molded article may contain, as the polypeptide, a polypeptide other than the spider silk polypeptide, and examples of the above-described polypeptide include silk fibroin, soybean protein, casein, keratin, collagen, and milk whey protein.

The molded article can be obtained by introducing the above-described composition into a casting mold (mold) and carrying out forming or the like, and, in the forming step, heating and/or pressing is possible. The composition has, typically, a shape of a powder form (lyophilized powder or the like) or a fibrous shape (a fiber or the like obtained by spinning), and the molded article may be a fused article of a composition comprising a polypeptide having the above-described shape.

Examples of the natural spider silk protein that is used as the polypeptide constituting the molded article include a major dragline silk protein and a flagelliform silk protein.

The major dragline silk protein is produced from spider's major ampullate glands and has a characteristic of excellent toughness. Examples of the major dragline silk protein include major ampullate spidroin MaSp1 or MaSp2 derived from Nephila clavipes, ADF3 or ADF4 derived from Araneus diadematus, and the like.

The flagelliform silk protein is produced from spider's flagelliform glands, and examples of the flagelliform silk protein include flagelliform silk protein derived from Nephila clavipes.

Examples of the polypeptides derived from natural spider silk protein include recombinant spider silk protein, for example, mutants, analogues, derivatives, and the like of natural spider silk protein. The above-described polypeptide is particularly preferably the recombinant spider silk protein of the major dragline silk protein.

As the polypeptide, in addition to the above-described spider silk polypeptide, silk fibroin, soybean protein, casein, keratin, collagen, or milk whey protein can be jointly used. The amount of silk protein or the like in the case of being jointly used needs to be an amount at which the transparency of the molded article can be ensured and is, for example, preferably 20 parts by mass or less, preferably 10 parts by mass or less, and more preferably 5 parts by mass or less with respect to 100 parts by mass of the spider silk polypeptide.

As the silk fibroin, it is possible to use sericin-removed silk fibroin and/or sericin-unremoved silk fibroin. Silk is a fiber that is obtained from cocoons made by silkworms that are larvae of Bombyx mori, and a cocoon filament is a single filament formed of two fibroin threads being covered with a gluey substance (sericin) on the outside. Fibroin is constituted of a number of fibrils, and the outside of fibroin is covered with four layers of sericin, thereby constituting one cocoon filament. Practically, sericin on the outside is dissolved and removed by means of refining, and cocoon filaments are used in clothing uses as silk filaments. The specific weight of silk is 1.33, the average fineness is 3.3 decitex, and the fiber length is, generally, approximately 1,300 to 1,500 m.

The silk fibroin is preferably silk fibroin lyophilized powder obtained by using cocoons of natural or domesticated silkworm or used or discarded silk cloth as a raw material and purifying silk fibroin from which sericin covering silk fibroin, other fat components, and the like are removed (lyophilized powder of sericin-removed silk fibroin). Meanwhile, as the silk fibroin, silk fibroin from which sericin and the like are not removed (sericin-unremoved silk fibroin) can also be used.

The molded article may be a molded article including the polypeptide alone and also may be a molded article in which additive components (for example, a plasticizer, a nucleating agent, an antioxidant, a coloring agent, a filler such as lamellar silicate or basic calcium phosphate, water, synthetic resins, and the like) are added to the polypeptide. In a case in which additive components of a plasticizer and the like are used, it is preferable to set the amount thereof to 50% by mass or less of the total amount of the polypeptides. In addition, foreign substances that are generated in a process of obtaining the polypeptide may also be included. Meanwhile, the molded article exhibits the effects of the present invention even in a case in which the above-described additive components are not added thereto.

The molded article has transparency. The transparency can be visually determined, but the molded article preferably has a transmittance of 50% or more in a case in which an optical transmittance measurement instrument is used and, for example, the cumulative time is set to 0.1 seconds in a wavelength range of 220 to 800 nm.

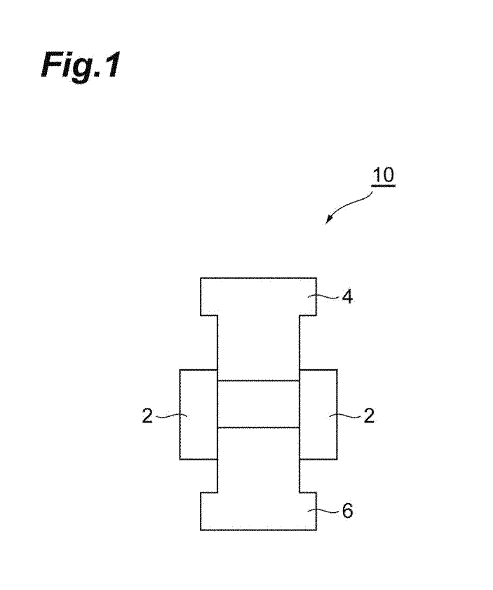

The molded article can be produced using a press molding machine. FIG. 1 is a schematic cross-sectional view of a press molding machine that can be used to produce the molded article. A press molding machine 10 illustrated in FIG. 1 includes a die 2 in which a through hole is formed and which can be heated and an upper side pin 4 and a lower side pin 6 which can be moved up and down in the through hole of the die 2. The molded article can be obtained by introducing the composition including the spider silk polypeptide into a cavity formed by inserting the upper side pin 4 or the lower side pin 6 into the die 2, and pressing the composition with the upper side pin 4 and the lower side pin 6 while heating the die 2.

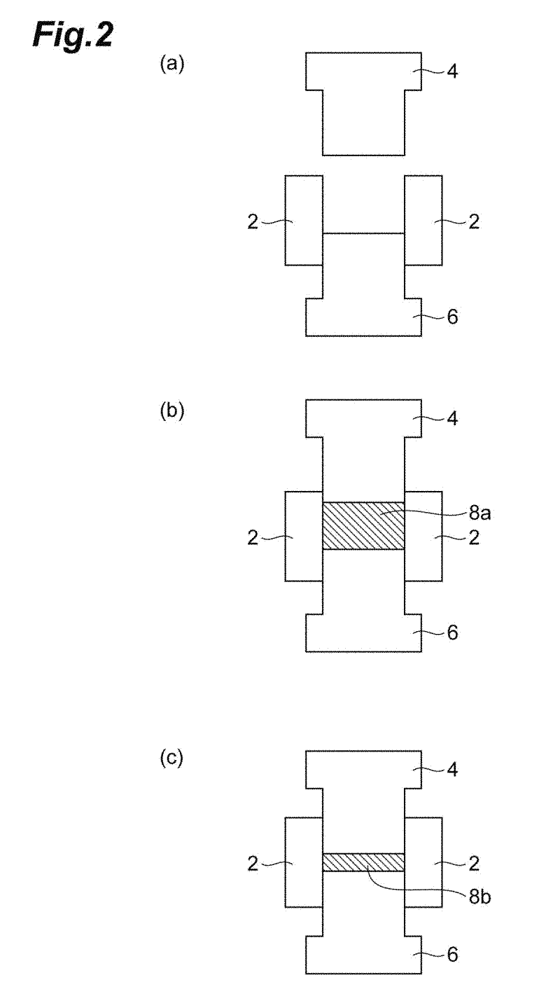

FIG. 2 illustrates step views of obtaining the molded article and are schematic cross-sectional views of the press molding machine (a) before the introduction of the composition, (b) immediately after the introduction of the composition, and (c) with the composition in a state of being heated and pressed. As illustrated in FIG. 2(a), the composition is introduced into the through hole in a state in which only the lower side pin 6 is inserted into the through hole of the die 2, and, as illustrated in FIG. 2(b), the upper side pin 4 is inserted into the through hole of the die 2 and moved down, and the heating of the die 2 is initiated, thereby heating and pressing a composition 8a which is not yet heated and pressed in the through hole. The upper side pin 4 is moved down until a predetermined pressurization force is reached, and heating and pressing are continued until the composition reaches a predetermined temperature in a state illustrated by FIG. 2(c), thereby obtaining a composition 8b which has been hot pressed. After that, the temperature of the die 2 is lowered using a cooler (for example, a spot cooler), the upper side pin 4 or the lower side pin 6 is removed from the die 2 when the composition 8b reaches a predetermined temperature, and the resulting content is taken out, thereby obtaining a molded article. The pressing may be carried out by moving down the upper side pin 4 in a state in which the lower side pin 6 is fixed, but it is also possible to move down the upper side pin 4 and move up the lower side pin 6 at the same time.

The heating is preferably carried out at 80° C. to 300° C., more preferably carried out at 100° C. to 180° C., and still more preferably carried out at 100° C. to 130° C. The pressing is preferably carried out at 5 kN or more, more preferably carried out at 10 kN or more, and still more preferably carried out at 20 kN or more. In addition, the time during which the treatment is continued under predetermined hot pressing conditions after the conditions are satisfied (temperature retention condition) is preferably 0 to 100 minutes, more preferably 1 to 50 minutes, and still more preferably 5 to 30 minutes.

The molded article of the composition comprising the polypeptide is preferably produced using the polypeptide derived from natural spider silk protein, and thus the production method will be described below in detail.

Examples of the polypeptide derived from the major dragline silk protein which serves as a raw material of the molded article include polypeptides including two or more, preferably including five or more, and more preferably including 10 or more units of an amino-acid sequence represented by Formula 1: REP1-REP2 (1). Alternatively, the polypeptide derived from the major dragline silk protein may be a polypeptide which includes a unit of the amino-acid sequence represented by Formula 1: REP1-REP2 (1) and in which a C terminal sequence is an amino-acid sequence represented by any of SEQ ID Nos. 1 to 3 or an amino-acid sequence having 90% or more homology with the amino-acid sequence represented by any of SEQ ID Nos. 1 to 3. Meanwhile, in the polypeptide derived from the major dragline silk protein, the units of the amino-acid sequence represented by Formula 1: REP1-REP2 (1) may be identical to or different from each other. In a case in which the production of recombinant protein is carried out using a microbe such as Escherichia coli as a host, the molecular weight of the polypeptide derived from the major dragline silk protein is preferably 500 kDa or less, more preferably 300 kDa or less, and still more preferably 200 kDa or less from the viewpoint of productivity.

In Formula 1, REP1 represents polyalanine. In REP1, alanines that are continuously arranged are preferably two or more residues, more preferably three or more residues, still more preferably four or more residues, and particularly preferably five or more residues. In addition, in REP1, alanines that are continuously arranged are preferably 20 or less residues, more preferably 16 or less residues, still more preferably 12 or less residues, and particularly preferably 10 or less residues. In Formula 1, REP2 is an amino-acid sequence made of 10 to 200 residues of amino acid, and the total residue number of glycine, serine, glutamine, and alanine in the amino-acid sequence is 40% or more, preferably 60% or more, and more preferably 70% or more of the total number of amino acid residues.

In the major dragline silk protein, REP1 corresponds to a crystalline region that forms a crystalline β sheet in the fiber, and REP2 corresponds to an amorphous region which is more flexible and lacks a regular structure in a majority area in the fiber. In addition, [REP1-REP2] corresponds to a repetitive region made up of crystalline regions and amorphous regions (repetitive sequence) and is the characteristic sequence of dragline silk protein.

An amino-acid sequence represented by SEQ ID No. 1 is the same as an amino-acid sequence made of 50 residues of amino acid at the C terminal of an amino-acid sequence of ADF3 (NCBI accession number: AAC47010, GI: 1263287), and an amino-acid sequence represented by SEQ ID No. 2 is the same as an amino-acid sequence obtained by removing 20 residues from the C terminal of the amino-acid sequence represented by SEQ ID No. 1, and an amino-acid sequence represented by SEQ ID No. 3 is the same as an amino-acid sequence obtained by removing 29 residues from the C terminal of the amino-acid sequence represented by SEQ ID No. 1.

As the polypeptide including two or more units of the amino-acid sequence represented by Formula 1: REP1-REP2 (1), it is possible to use, for example, a polypeptide made of an amino-acid sequence represented by SEQ ID No. 7. The polypeptide made of the amino-acid sequence represented by SEQ ID No. 7 is an amino-acid sequence of ADF3 (NCBI accession number: AAC47010, GI: 1263287) having an amino-acid sequence (SEQ ID No. 4) made up of an initiation codon, a His10 tag, and a Human rhinovirus 3C protease (HRV3C protease) recognition site added to an N terminal which is mutated so that translation ends at the 543^{rd} amino acid residue.

In addition, as the polypeptide including two or more units of the amino-acid sequence represented by Formula 1: REP1-REP2 (1), it is possible to use a protein which is made of the amino-acid sequence represented by SEQ ID No. 7 in which one or a plurality of amino acids is substituted, deleted, inserted, and/or added, and has a repetitive region made up of crystalline regions and amorphous regions. In the present invention, “one or a plurality” means, for example, 1 to 40, 1 to 35, 1 to 30, 1 to 25, 1 to 20, 1 to 15, 1 to 10, or 1 or several. In addition, in the present invention, “one or several” means 1 to 9, 1 to 8, 1 to 7, 1 to 6, 1 to 5, 1 to 4, 1 to 3, 1 or 2, or 1.

In addition, examples of the polypeptide including two or more units of the amino-acid sequence represented by Formula 1: REP1-REP2 (1) include ADF4-derived recombinant protein having an amino-acid sequence represented by SEQ ID No. 8. The amino-acid sequence represented by SEQ ID No. 8 is an amino-acid sequence obtained by adding the amino-acid sequence (SEQ ID No. 4) made up of an initiation codon, a His10 tag, and a Human rhinovirus 3C protease (HRV3C protease) recognition site to an N terminal of a partial amino-acid sequence of ADF4 (NCBI accession number: AAC47011, GI: 1263289) procured from NCBI database. In addition, as the polypeptide including two or more units of the amino-acid sequence represented by Formula 1: REP1-REP2 (1), a polypeptide which is made of the amino-acid sequence represented by SEQ ID No. 8 in which one or a plurality of amino acids is substituted, deleted, inserted, and/or added, and has a repetitive region made up of crystalline regions and amorphous regions may be used. In addition, examples of the polypeptide including two or more units of the amino-acid sequence represented by Formula 1: REP1-REP2 (1) include MaSp2-derived recombinant protein having an amino-acid sequence represented by SEQ ID No. 9. The amino-acid sequence represented by SEQ ID No. 9 is an amino-acid sequence obtained by adding an amino-acid sequence made up of an initiation codon, a His10 tag, and a Human rhinovirus 3C protease (HRV3C protease) recognition site to an N terminal of a partial amino-acid sequence of MaSp2 (NCBI accession number: AAT75313, GI: 50363147) procured from NCBI database. In addition, as the polypeptide including two or more units of the amino-acid sequence represented by Formula 1: REP1-REP2 (1), a polypeptide which is made of the amino-acid sequence represented by SEQ ID No. 9 in which one or a plurality of amino acids is substituted, deleted, inserted, and/or added, and has a repetitive region made up of crystalline regions and amorphous regions may also be used.

Examples of the polypeptides derived from the flagelliform silk protein include polypeptides including 10 or more, preferably including 20 or more, and more preferably including 30 or more units of an amino-acid sequence represented by Formula 2: REP3 (2). In a case in which the production of recombinant protein is carried out using a microbe such as Escherichia coli as a host, the molecular weight of the polypeptide derived from the flagelliform silk protein is preferably 500 kDa or less, more preferably 300 kDa or less, and still more preferably 200 kDa or less from the viewpoint of productivity.

In Formula (2), REP3 refers to an amino-acid sequence constituted of Gly-Pro-Gly-Gly-X, and X refers to one amino acid selected from the group consisting of Ala, Ser, Tyr, and Val.

In spider silk, flagelliform silk has a significant characteristic of having a repetitive region which does not have any crystalline regions and is made of an amorphous region. Major dragline silk and the like have a repetitive region made up of crystalline regions and amorphous regions and are thus assumed to have both a high stress and a high stretching property. On the other hand, flagelliform silk has a stress that is poorer than that of major dragline silk, but has a favorable stretching property. This is considered to be due to the fact that a majority of flagelliform silk is constituted of an amorphous region.

Examples of the polypeptide including 10 or more units of the amino-acid sequence represented by Formula 2: REP3 (2) include flagelliform silk protein-derived recombinant protein having an amino-acid sequence represented by SEQ ID No. 10. The amino-acid sequence represented by SEQ ID No. 10 is an amino-acid sequence obtained by combining an amino-acid sequence of the 1220^{th} residue through the 1659^{th} residue from an N terminal that corresponds to a repeat portion and a motif of a partial sequence of a flagelliform silk protein of Nephila clavipes (NCBI accession number: AAF36090, GI: 7106224) procured from NCBI database (referred to as the PR1 sequence) and a C terminal amino-acid sequence of the 816^{th} residue through the 907^{th} residue from a C terminal of a partial sequence of a flagelliform silk protein of Nephila clavipes (NCBI accession number: AAC38847, GI: 2833649) procured from NCBI database and adding the amino-acid sequence (SEQ ID No. 4) made up of an initiation codon, a His10 tag, and an HRV3C protease recognition site to an N terminal of the combined sequence. In addition, as the polypeptide including 10 or more units of the amino-acid sequence represented by Formula 2: REP3 (2), a polypeptide which is made of the amino-acid sequence represented by SEQ ID No. 10 in which one or a plurality of amino acids is substituted, deleted, inserted, and/or added, and has a repetitive region made of an amorphous region may also be used.

The polypeptide can be produced using a host that is transformed by an expression vector containing a gene that codes the polypeptide. A method for producing the gene is not particularly limited, and the gene is produced by amplifying a gene that codes natural spider silk protein from a spider-derived cell by means of a polymerase chain reaction (PCR) or the like and cloning the gene or is chemically synthesized. A method for chemically synthesizing the gene is not particularly limited, and, for example, the gene can be synthesized by linking oligonucleotides that are automatically synthesized with AKTAoligopilot plus 10/100 (manufactured by GE Healthcare Company Japan) or the like by means of PCR on the basis of the amino-acid sequence information of natural spider silk protein procured from the web database of NCBI or the like. At this time, in order to facilitate the refining or confirmation of protein, a gene that codes a protein made of an amino-acid sequence obtained by adding an amino-acid sequence made up of an initiation codon and a His10 tag to an N terminal of the amino-acid sequence may be synthesized.

As the expression vector, it is possible to use a plasmid, a phage, a virus, or the like which is capable of expressing proteins from DNA sequences. The plasmid-type expression vector is not particularly limited as long as the expression vector is capable of expressing the target gene in host cells and is self-amplifiable. For example, in a case in which Escherichia coli Rosetta (DE3) is used as a host, it is possible to use a pET22b(+) plasmid vector, a pCold plasmid vector, or the like. Among these, from the viewpoint of the productivity of protein, the pET22b(+) plasmid vector is preferably used. As the host, it is possible to use, for example, an animal cell, a plant cell, a microbe, or the like.

The polypeptide that is used in the present invention is preferably a polypeptide derived from ADF3 which is one of the two important dragline silk proteins of Araneus diadematus. This polypeptide, basically, has a high strength-elongation degree and a high toughness and also has an advantage of ease of synthesis.

### EXAMPLES

Hereinafter, the present invention will be described in more detail using examples, but the present invention is not limited to these examples within the scope of the technical concept of the present invention.

<Gene Synthesis>

(1) Synthesis of Gene of ADF3Kai

A partial amino-acid sequence of ADF3 which is one of the two important dragline silk proteins of Araneus diadematus (NCBI accession number: AAC47010, GI: 1263287) was acquired from the web database of NCBI, and the synthesis of a gene that codes an amino-acid sequence (SEQ ID No. 5) obtained by adding (SEQ ID No. 4) made up of an initiation codon, a His10 tag, and a Human rhinovirus 3C protease (HRV3C protease) recognition site to an N terminal of the same sequence was entrusted to GenScript Japan Inc. As a result, a pUC57 vector (having an Nde I site at right upstream region of a 5′ terminal of the gene and an Xba I site at right downstream region of the 5′ terminal) into which a gene of ADF3Kai made of a base sequence represented by SEQ ID No. 6 was introduced was acquired. Then, a restriction enzyme treatment was carried out on the same gene using Nde I and EcoR I, and the gene was recombined to a pET22b(+) expression vector.

(2) Synthesis of Gene of ADF3Kai-noNR

The above-obtained pET22b(+) expression vector into which the gene of ADF3Kai was introduced was used as a template, and a codon GTG corresponding to the 543^{rd} amino acid residue valine (Val) in the amino-acid sequence (SEQ ID No. 5) of ADF3Kai was mutated to a termination codon TAA by means of site-directed mutagenesis using PrimeStar MutagenesisBasal Kit (manufactured by Takara Bio Inc.), thereby constructing a gene of ADF3Kai-noNR represented by SEQ ID No. 11 on pET22b(+). The accuracy of the mutagenesis was confirmed from a sequence reaction for which 3130×1 Genetic Analyzer (Applied Biosystems) was used. Meanwhile, the amino-acid sequence of ADF3Kai-noNR is as indicated by SEQ ID No. 7.

<Expression of Protein>

The pET22b(+) expression vector including a gene sequence of the above-obtained ADF3Kai-noNR was transformed to Escherichia coli Rosetta (DE3). The obtained single colony was cultured for 15 hours in a 2 mL LB culture medium including ampicillin, 1.4 mL of the resulting culture liquid was added to a 140 mL LB culture medium including ampicillin, and the culture liquid was cultured under conditions of 37° C. and 200 rpm until OD₆₀₀ of the culture liquid reached 3.5. Next, the culture liquid having OD₆₀₀ of 3.5 was added to a 7 L 2×YT culture medium including ampicillin together with 140 mL of 50% glucose and was further cultured until OD₆₀₀ reached 4.0. Then, isopropyl-β-thiogalactopyranoside (IPTG) was added to the obtained culture liquid having OD₆₀₀ of 4.0 so that the final concentration reached 0.5 mM, thereby inducing protein expression. After two hours elapsed from the addition of IPTG the culture liquid was centrifugally separated, and a bacterial body was collected. As a result of electrophoresing protein solutions prepared from the culture liquid before that addition of IPTG and after the addition of IPTG in polyacrylamide gels, bands having a target size were observed depending on the addition of IPTG, and it was confirmed that target protein was expressed. Escherichia coli that expressed the protein of ADF3Kai-noNR was stored in a freezer (−20° C.).

<Polypeptide Preparation Example>

(I) Approximately 4.5 g of the bacterial body of Escherichia coli that expressed the protein of ADF3Kai-noNR and 30 mL of a buffer solution AI (20 mM of Tris-HCl, pH 7.4) were added to a centrifuge tube (50 mL), the bacterial body was dispersed using a mixer (manufactured by General Electric, SI-0286, level 10), then, centrifugal separation (10,000 rpm, 10 minutes, room temperature) was carried out using a centrifugal separator (manufactured by Tomy Seiko Co., Ltd., MX-305), and the supernatant was removed.

(II) 30 mL of a buffer solution AI and 0.3 mL of 0.1 M PMSF (dissolved using isopropanol) were added to the precipitate (bacterial body) obtained by the centrifugal separation and were dispersed for three minutes using the mixer (level 10) manufactured by General Electric. After that, the bacterial body was crushed using an ultrasonic crusher (manufactured by Sonic & Materials, Inc., VCX500), and centrifugal separation (10,000 rpm, 10 minutes, room temperature) was carried out.

(III) 30 mL of the buffer solution AI was added to the precipitate obtained by the centrifugal separation, was dispersed for three minutes using a mixer (manufactured by IKA, T18 basic ULTRA-TURRAX, level 2) manufactured by General Electric, then, centrifugal separation (10,000 rpm, 10 minutes, room temperature) was carried out using the centrifugal separator manufactured by Tomy Seiko Co., Ltd., and the supernatant was removed.

(IV) A 7.5 M urea buffer solution I (7.5 M of urea, 10 mM of sodium dihydrogen phosphate, 20 mM of NaCl, 1 mM of Tris-HCl, pH 7.0) was added to the centrifuge tube from which the supernatant was removed, and the precipitation was favorably dispersed using the ultrasonic crusher manufactured by SMT (level 7). After that, the precipitate was dissolved for 120 minutes using a shaker manufactured by Taitec Corporation (200 rpm, 60° C.). A protein solution after the dissolution was centrifugally separated (11,000×g, 10 minutes, room temperature) using the centrifugal separator manufactured by Tomy Seiko Co., Ltd., and the supernatant was dialyzed using a dialysis tube (manufactured by Sanko Junyaku Co., Ltd., cellulose tube 36/32). White agglomerated protein obtained after the dialysis was collected by means of centrifugal separation, water was removed using a freeze dryer, and freeze-dried powder was collected. The degree of refining of the target protein ADF3Kai-noNR in the obtained freeze-dried powder was confirmed by image-analyzing the results of the polyacrylamide gel electrophoresis (CBB dyeing) of the powder using Totallab (Nonlinear Dynamics Ltd.). As a result, the degree of refining of ADF3Kai-noNR was approximately 85%.

### Example 1

<Method for Producing Molded Article>

1.35 g of the freeze-dried powder obtained in the “polypeptide preparation example” (hereinafter, referred to as “sample”) was weighed, and this sample was introduced into the through hole of the die 2 (a cylindrical die having a rectangular through hole having a 35 mm×15 mm cross section) of the press molding machine 10 illustrated in FIG. 1. At this time, the sample was added so that it became uniform thickness. After the entire sample was introduced, the heating of the die 2 was initiated, and the upper side pin 4 and the lower side pin 6 were inserted into the through hole using a hand presser (manufactured by NPa System Co., Ltd., NT-100H-V09), thereby pressing the sample. At this time, the pressurization condition of the sample was controlled to be 40 kN. The heating was stopped when the temperature of the sample reached 200° C., the sample was cooled using a spot cooler (manufactured by Trusco Nakayama Corporation, TS-25EP-1) and was removed when the temperature of the sample reached 50° C., and deburring was carried out, thereby obtaining a 35 mm×15 mm×2 mm rectangular molded article.

<Methods for Measuring Bending Elastic Modulus and Bending Strength>

After the obtained molded article was left to stand for one day in a constant temperature and humidity tank (manufactured by ESPEC Corp., LHL-113) under conditions of 20° C./65% RH, the following measurements were carried out.

That is, a three-point bending test was carried out using a basket jig in an autograph (manufactured by Shimadzu Corporation, AG-X plus). A load cell used was 50 kN. At this time, the distance between the support points for three-point bending was fixed to 27 mm, and the measurement rate was set to 1 mm/minute. In addition, the size of the molded article was measured using a microcaliper, and the molded article was installed in the jig and was measured. The bending elastic modulus was obtained from a displacement (strain) of up to 0.05 to 0.25%.

<Method for Measuring Transparency 1>

For the obtained molded articles, the transparency in the thickness direction (2 mm-thick) was visually measured, and molded articles that were highly transparent as a whole were evaluated as A, molded articles that were transparent, but included a small number of semitransparent or opaque portions were evaluated as B, and molded articles that were not transparent were evaluated as C. That is, the transparency was confirmed from whether or not the logo was visible when the molded article was placed on paper on which the logo of Spiber Inc. was printed.

### Comparative Examples 1 and 2

35 mm×15 mm×2 mm rectangular formed articles were obtained in the same manner as in Example 1 using polycarbonate (PC) and poly(methyl methacrylate) (PMMA). These formed articles were left to stand for one day in the constant temperature and humidity tank (manufactured by ESPEC Corp., LHL-113) under conditions of 20° C./65% RH, and then the bending elastic moduli, the bending strengths, and the transparency were measured in the same manner as in Example 1.

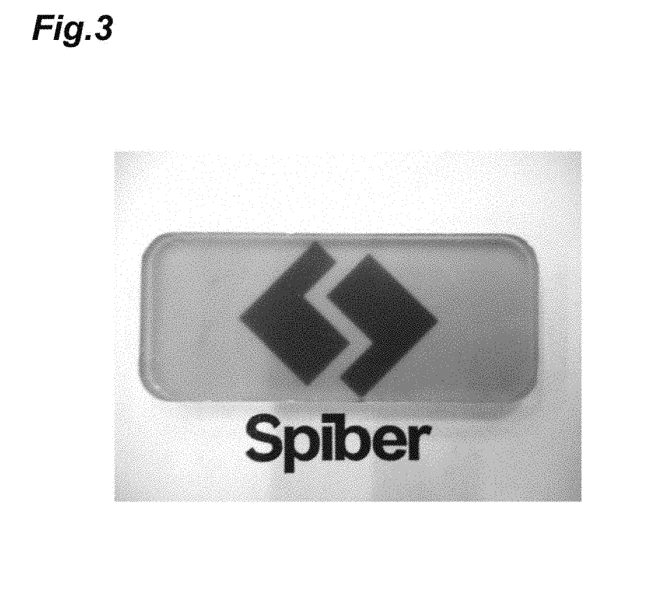

The results of the example and the comparative examples are summarized in Table 1. A photograph of the molded article used to evaluate the transparency of Example 1 is illustrated in FIG. 3.

TABLE 1

| | Bending Elastic | Bending | |
|---|---|---|---|
| | Modulus | Strength | Transpar- |
| | (GPa) | (MPa) | ency |
| Example 1 | 5.39 | 74 | A |
| Comparative Example 1 | 2.68 | 97.3 | A |
| (PC) | | | |
| Comparative Example 2 | 3.2 | 105 | A |
| (PMMA) | | | |

### Examples 2 to 18

In Example 1, the heating was stopped when the temperature of the sample reached 200° C., the sample was cooled using the spot cooler and was taken out when the temperature of the sample reached 50° C. That is, the heating temperature (X) was 200° C., and the annealing time (Y) was 0 minutes since the rapid cooling was initiated immediately after the heating temperature was reached. Molded articles were obtained in the same manner as in Example 1 except for the fact that the heating temperature (X) and the annealing time (Y) were changed as shown in Table 2 and the pressurization condition was set to 30 kN. For the obtained molded articles, their transparencies were evaluated in the same manner as in Example 1 and were summarized in Table 2.

TABLE 2

| | Heating | Annealing | |
|---|---|---|---|
| | Temperature X | Time Y | Transpar- |
| | (° C.) | (Minutes) | ency |
| Example 2 | 110 | 0 | B |
| Example 3 | 120 | 0 | B |
| Example 4 | 130 | 0 | A |
| Example 5 | 140 | 0 | A |
| Example 6 | 160 | 0 | A |
| Example 7 | 180 | 0 | A |
| Example 8 | 90 | 5 | B |
| Example 9 | 100 | 5 | A |
| Example 10 | 110 | 5 | A |
| Example 11 | 120 | 5 | A |
| Example 12 | 90 | 15 | B |
| Example 13 | 120 | 15 | A |
| Example 14 | 80 | 30 | B |
| Example 15 | 90 | 30 | A |
| Example 16 | 100 | 30 | A |
| Example 17 | 110 | 30 | A |
| Example 18 | 120 | 30 | A |

### Examples 19 to 24

Molded articles were obtained in the same manner as in Example 1 except for the fact that the heating temperature (X), the annealing time (Y), and the pressurization condition were set as shown in Table 3. For the obtained molded articles, their transparencies were evaluated in the same manner as in Example 1 and were summarized in Table 3.

TABLE 3

| | Heating | Annealing | Pressing | |
|---|---|---|---|---|
| | Temperature X | Time Y | Condition | Transpar- |
| | (° C.) | (Minutes) | (kN) | ency |
| Example 19 | 90 | 0 | 40 | B |
| Example 20 | 100 | 0 | 40 | A |
| Example 21 | 110 | 0 | 40 | A |
| Example 22 | 120 | 0 | 40 | A |
| Example 23 | 90 | 0 | 50 | B |
| Example 24 | 100 | 0 | 50 | A |

For Examples 16 to 18 and 5 to 7, the bending elastic moduli and the bending strengths were measured in the same manner as in Example 1, and the transmittance was measured on the basis of the following method for measuring transparency 2. The results are shown in Table 4.

<Method for Measuring Transparency 2>

The transmittance was obtained using an UV-Vis spectrophotometer (manufactured by Shimadzu Corporation, UV-2600) in a wavenumber range of 220 to 800 nm for a cumulative time of 0.1 seconds.

TABLE 4

| | Heating | | Bending | | |
|---|---|---|---|---|---|
| | Temper- | Annealing | Elastic | Bending | |
| | ature X | Time Y | Modulus | Strength | Iransmittance |
| | (° C.) | (Minutes) | (GPa) | (MPa) | (%) |
| Example | 100 | 30 | 6.6 | 64.6 | 54.1 |
| 16 | | | | | |
| Example | 110 | 30 | 6.3 | 67.1 | 54.5 |
| 17 | | | | | |
| Example | 120 | 30 | 5.9 | 62.5 | 56.6 |
| 18 | | | | | |
| Example | 140 | 0 | 5.2 | 52.1 | 53.8 |
| 5 | | | | | |
| Example | 160 | 0 | 5.8 | 56.7 | 54.3 |
| 6 | | | | | |
| Example | 180 | 0 | 5.7 | 63.5 | 55.2 |
| 7 | | | | | |

### REFERENCE SIGNS LIST

2: DIE, 4: UPPER SIDE PIN, 6: LOWER SIDE PIN, 8a: COMPOSITION BEFORE HOT PRESSING, 8b: COMPOSITION AFTER HOT PRESSING, 10: PRESS MOLDING MACHINE

1. A molded article of a composition comprising a polypeptide, wherein the molded article has more than 4.3 GPa of a bending elastic modulus and transparency.

2. The molded article according to claim 1, wherein the polypeptide comprises at least one kind selected from the group consisting of natural spider silk protein and a polypeptide derived from natural spider silk protein.

3. The molded article according to claim 1, wherein the molded article is a hot press-molded article.

4. A method for producing a molded article for producing the molded article according to claim 1, the method comprising a step of hot pressing the composition comprising the polypeptide.

5. The molded article according to claim 2, wherein the molded article is a hot press-molded article.

6. A method for producing a molded article for producing the molded article according to claim 2, the method comprising a step of hot pressing the composition comprising the polypeptide.

7. A method for producing a molded article for producing the molded article according to claim 3, the method comprising a step of hot pressing the composition comprising the polypeptide.

8. A method for producing a molded article for producing the molded article according to claim 5, the method comprising a step of hot pressing the composition comprising the polypeptide.
